(19) United States
(12) Patent Application Publication
(10) Pub. No.: US 2009/0208458 A1
(43) Pub. Date: Aug. 20, 2009

(54) Transcriptional inhibitor for human k-ras gene

(75) Inventors: Masanori Hatakeyama, Hokkaido (JP); Masatomo Yanagihara, Hokkaido (JP)

Correspondence Address: EDWARDS ANGELL PALMER & DODGE LLP, P.O. BOX 55874, BOSTON, MA, 02205, US

(73) Assignee: National University Corporation Hokkaido University, Hokkaido (JP)

(21) Appl. No.: 11/666,520

(22) PCT Filed: Jul. 1, 2005
(86) PCT No.: PCT/JP05/12204

§ 371 Date: Dec. 11, 2007

(30) Foreign Application Priority Data
Oct. 29, 2004 (JP) 2004-315291

(51) Int. Cl. A61K 35/76; C07K 14/00; C12N 15/11; C12N 15/00; A61K 38/16

(52) U.S. Cl. 424/93.2; 530/350; 536/23.1; 435/320.1; 514/12

(57) ABSTRACT

A transcriptional inhibitor for human K-ras gene which comprises one or more proteins selected from the group consisting of a protein having the amino acid sequence represented by SEQ ID NO:1, a protein having an amino acid sequence derived from the amino acid sequence represented by SEQ ID NO:1 by substitution, deletion or addition of one to several amino acids and having an activity of inhibiting the transcription of human K-ras gene, and partial fragment proteins thereof having an activity of inhibiting the transcription of human K-ras gene. This transcriptional inhibitor for human K-ras gene specifically inhibits the transcription and expression of K-ras gene, which is an oncogene, in a human cancer cell. Thus, it can inhibit the proliferation of cancer cells and induce the reversion of cancer cells into normal cells, which makes it usable as an anticancer agent with little side effects on normal cells.

### TECHNICAL FIELD

The invention relates to a transcriptional inhibitor for human K-ras gene capable of specifically inhibiting the expression of K-ras gene which is a human oncogene, particularly the expression of K-ras oncogene, and also relates to a protein having an activity of inhibiting the transcription of human K-ras gene.

### BACKGROUND ART

Ras represents oncogenes identified, for the first time, as genes causing oncogenic virus-induced malignant tumors (Non-Patent Documents 1 and 2) and constitute the Ras gene family (H-Ras, K-Ras, N-Ras, R-Ras).

Ras genes encode GTP-binding proteins with a molecular weight of 21 kDa, and any of them are activated from the GDP binding form to the GTP binding form through a growth factor receptor to transmit a signal to the MAP kinase cascade and thus involved in cell growth or differentiation.

It is known that Ras genes (proteins) have no carcinogenic activity by themselves but can acquire carcinogenic activity when mutated, so that cells having the mutation can be transformed (Non-Patent Document 3). Until now, Ras gene mutations in many types of human cancer cells are reported (Non-Patent Document 4).

K-ras gene, a member of the Ras gene family, is located on the short arm of human chromosome 12. Its activated mutant (K-ras oncogene) is one of the oncogenes most frequently found in human cancers and observed to exist in about 50% of human colon cancers, 25 to 50% of lung cancers, and 70 to 90% of pancreatic cancers (Non-Patent Documents 5 and 6). It has been revealed that in these cancers, continuous expression and production of mutated K-Ras protein from K-ras oncogene are essential for the canceration of cells and the maintenance of the characteristics of cancer cells.

Thus, there have been attempts to inhibit cancers by inhibiting in vivo the expression of ras genes, typically K-ras gene, particularly K-ras oncogene, or by inhibiting in vivo the function of K-ras oncogene products.

Known examples include the inhibition of RAS gene with an antisense oligonucleotide against RAS gene (Patent Document 1) and an agent for treating pancreatic cancer which uses an effective amount of farnesylamine, geranylgeranylamine, or a derivative thereof (Patent Document 2).

The invention is to provide a proteinaceous pharmaceutical capable of inhibiting the expression of K-ras gene, particularly K-ras oncogene.

Patent Document 1: WO99/02732

Patent Document 2: Japanese Patent Application Laid-Open (JP-A) No. 10-218764

Non-Patent Document 1: Barbacid, M., Annu. Rev. Biophem. 56, 779-827(1987)

Non-Patent Document 2: Lowy, D. R. & Willumsen, B. M., Annu. Rev. Biophem. 62, 851-891(1993)

Non-Patent Document 3: Storer R D et al., Cancer Res. 46:1458-1464, 1986

Non-Patent Document 4: Minamoto T et al., Cancer Detection and Prevention 24:1-12, 2000

Non-Patent Document 5: Clark, G. J. & Der, C. J., Cellular Cancer Markers (eds Garrett, C. T. & Sell, S.)17-52 (Humana Press, Totowa, N.J. (1995))

Non-Patent Document 6: Bos, J. L., Cancer Res. 49, 4682-4689(1989)

### DISCLOSURE OF THE INVENTION

The inventors have made investigations on the physiological function of a homeodomain-containing protein that comprises the amino acid sequence shown in SEQ ID NO:1 and is determined to be expressed in human testes (hereinafter referred to as ESXR1). As a result, the inventors have unexpectedly found that ESXR1 and an N-terminal fragment thereof (hereinafter referred to as ESXR1-ΔC) have an activity in vivo to specifically inhibit transcription of K-ras gene in cancer cells and have completed each of the following aspects of the invention:

1) A transcriptional inhibitor for human K-ras gene, comprising one or more proteins selected from the group consisting of a protein comprising an amino acid sequence shown in SEQ ID NO:1, a protein comprising an amino acid sequence derived from the amino acid sequence shown in SEQ ID NO:1 by substitution, deletion or addition of one or several amino acids and having an activity of inhibiting the transcription of human K-ras gene, or a protein fragment thereof having an activity of inhibiting the transcription of human K-ras gene;

2) The transcriptional inhibitor for human K-ras gene according to Item 1), wherein the protein fragment is an N-terminal fragment with a molecular weight of 45 kd;

3) The transcriptional inhibitor for human K-ras gene according to Item 2), wherein the protein fragment has an amino acid sequence of residues 1 to 229 of SEQ ID NO:1 or an amino acid sequence derived from the amino acid sequence of residues 1 to 229 by substitution, deletion or addition of one or several amino acids;

4) A protein comprising any one of the amino acid sequences shown in SEQ ID NOS:2 to 4, or a protein derived from the protein comprising any one of the amino acid sequences shown in SEQ ID NOS:2 to 4 by substitution, deletion or addition of one or several amino acids and having an activity of inhibiting the transcription of human K-ras gene;

5) A nucleic acid encoding the protein according to Item 4);

6) A nucleic acid comprising a base sequence shown in any one of SEQ ID NOS:2 to 4 or a nucleic acid comprising a base sequence capable of hybridizing to any one of the base sequences shown in SEQ ID NOS:2 to 4 under stringent. conditions and encoding a protein having an activity of inhibiting the transcription of human K-ras gene;

7) A recombinant virus vector for use in gene therapy of cancer, comprising either a nucleic acid encoding the protein according to any one of Items 1) to 4) or the nucleic acid according to Item 5) or 6);

8) A recombinant vector, comprising the nucleic acid according to Item 5) or 6);

9) A method for treating cancer, comprising administering the transcriptional inhibitor for human K-ras gene according to any one of Items 1) to 3) or the protein according to Item 4) to cancer cells; and

10) A method for gene therapy of cancer, comprising introducing the recombinant virus vector according to Item 7) into cancer cells of a patient.

### BRIEF DESCRIPTION OF THE DRAWINGS

FIG. 1 shows agarose electrophoresis in a binding test between GST-ESXR1-HD and DNA having a P3 consensus region;

FIG. 2 shows radioautographs in binding tests between Myc-ESXR or Myc-ESXR1-ΔC and DNA having any P3 consensus region;

FIG. 3 shows the expression-inhibiting effect of ESXR1 on a reporter plasmid having any P3 consensus region;

FIG. 4 shows intracellular K-Ras protein expression levels in DOX-treated U2/tetESXR1 cells;

FIG. 5 shows intracellular K-Ras protein expression levels in DOX-treated U2-OS cells;

FIG. 6 shows K-Ras protein expression in U2/tetESXR1 cells arrested at the S phase and the G2/M phase;

FIG. 7 shows intracellular H-ras protein expression levels in DOX-treated U2/tetESXR1 cells;

FIG. 8 shows a reduction in the level of mRNA expression of intracellular K-ras gene by the expression of ESXR1;

FIG. 9 shows the expression-inhibiting effect of ESXR1 on a reporter plasmid having a P3 consensus region of the K-ras gene;

FIG. 10 shows the growth ability of colon cancer cells expressing ESXR1;

FIG. 11 shows the intracellular K-ras protein expression level of colon cancer cells expressing ESXR1; and

FIG. 12 shows the growth ability of colon cancer cells expressing ESXR1.

### BEST MODE FOR CARRYING OUT THE INVENTION

The transcriptional inhibitor for human K-ras gene of the invention comprises one or more proteins selected from the group consisting of a protein (ESXR1) comprising an amino acid sequence shown in SEQ ID NO:1, a protein comprising an amino acid sequence derived from the amino acid sequence shown in SEQ ID NO:1 by substitution, deletion or addition of one or several amino acids and having an activity of inhibiting the transcription of human K-ras gene, or fragments thereof having an activity of inhibiting the transcription of human K-ras gene.

ESXR1 is a protein comprising 406 amino acid residues in total, and it is reported by Fohn et al. that the gene (ESXR1) encoding it is expressed mainly in human testes (Fohn L. E. et al., Genomics, Vol. 74, pp. 105-108, 2001). This paper discloses that the nucleic acid sequence is characterized by having a homeobox domain but provides no information about the physiological function of the protein ESXR1.

As a result of the inventors' research, it is reported that: ESXR1 has the function of inhibiting cyclin degradation in human cells; ESXR1 is processed by an intracellular protease into an about 45 kd N-terminal fragment and an about 20 kd C-terminal fragment; the C-terminal fragment is involved in the cyclin degradation; the N-terminal fragment is localized in a nucleus; and so on (Ozawa et al., Oncogene, Vol. 23, 6590-6602, 2004).

Unexpectedly, it has been found that against the K-ras oncogene in human cancer cells, ESXR1-ΔC, an N-terminal fragment of ESXR1, recognizes and binds to TAATGTTATTA, which is a nucleic acid sequence in the first intron of the K-ras oncogene, so that it has an activity to specifically inhibit the expression thereof and to inhibit the growth of the cancer cells.

Thus, if ESXR1-ΔC or ESXR1 which can produce it by intracellular processing is administered to cancer cells, or if a gene encoding any of them is expressed in cancer cells, it will be possible to specifically inhibit the expression of the K-ras gene, in particular, the expression of the K-ras oncogene, in cancer cells. The specificity means that there will be observed no negative effect on normal cells other than cancer cells, and thus the transcriptional inhibitor for K-ras gene of the invention can provide a good anticancer drug with high selectivity for cancer cells.

Any polypeptide or protein comprising an amino acid sequence derived from the amino acid sequence shown in SEQ ID NO:1 or 2 by substitution, deletion and/or addition of one or more amino acids, or any transcriptional inhibitor for K-ras gene including the above falls within the scope of the invention, as long as it has K-ras gene transcription-inhibiting activity.

For proteins, it is empirically known that highly conservative variations of proteins are allowed with respect to physical and chemical properties such as the charge, size and hydrophobicity of amino acid residues. For example, amino acid residue substitution is possible between glycine (Gly) and proline (Pro), Gly and alanine (Ala) or valine (Val), leucine (Leu) and isoleucine (Ile), glutamic acid (Glu) and glutamine (Gln), asparatic acid (Asp) and asparagine (Asn), cysteine (Cys) and threonine (Thr), Thr and serine or Ala, or lysine (Lys) and arginine (Arg). Even beyond the conservation as stated above, one skilled in the art will experience any variation in which the essential function of proteins, the K-ras gene transcription-inhibiting activity in the case of the invention, still remains. In addition, many cases are also known in which the same type of proteins conservative between different organisms can maintain the essential function even when some amino acids are locally or dispersively deleted or inserted.

Thus, it will be understood that any protein comprising an amino acid sequence derived from the amino acid sequence shown in SEQ ID NO:1 or 2 by substitution, deletion and/or addition of one or more amino acids will fall within the scope of the invention as being the transcriptional inhibitor for K-ras gene if the protein has the K-ras gene transcription-inhibiting activity.

Such amino acid modifications may be observed in nature like genetic polymorphism and so on, and may be artificially made using methods known to one skilled in the art, such as mutagenesis techniques with mutagenic agents such as NTG and site-directed mutagenesis techniques based on various genetic recombination methods.

While the amino acid mutation may occur at any site or in any number as long as proteins having the K-ras gene transcription-inhibiting activity are provided, the number of mutated amino acids is generally several tens or less, preferably ten or less. The allowable range of modification may be indicated by the degree of amino acid sequence identity. According to that, the amino acid sequence of the protein of the invention may have a sequence identity of 80% or more, preferably of 90% or more, more preferably of 95% or more, with that shown in SEQ ID NO:1 or 2.

The nucleic acid of the invention may be a gene encoding a protein comprising any of the amino acid sequences shown in SEQ ID NOS:2 to 4, typically a nucleic acid comprising any of the base sequences shown in SEQ ID NOS:2 to 4. It will be understood that such a nucleic acid can be readily prepared based on the base sequences disclosed in SEQ ID NOS:2 to 4 by one skilled in the art using cloning by general genetic engineering techniques such as hybridization or chemical synthesis techniques such as phosphoramidite methods. Examples of the nucleic acid form include, but are not limited to, cDNA, genomic DNA and chemically synthesized DNA.

RNA sequences derivable from the base sequences shown in SEQ ID NOS:2 to 4 and DNA and RNA sequences complementary thereto can be uniquely determined. Thus, the invention also provides such RNAs and complementary DNAs or RNAs.

The nucleic acid of the invention also includes any nucleic acid that comprises a base sequence capable of hybridizing to any of the base sequences shown in SEQ ID NOS:2 to 4 under stringent conditions and encodes a protein having an activity of inhibiting the transcription of human K-ras gene.

The base sequence capable of hybridizing to the nucleic acid comprising the base sequence shown in any of SEQ ID NOS: 2 to 4, under stringent conditions, may be varied, as long as the protein encoded by the nucleic acid has K-ras gene transcription-inhibiting activity.

For example, the base sequence may be partially modified using different codons encoding the same amino acid residue (degenerate codons), a variety of artificial processes such as site-directed mutagenesis, random mutation by mutagenic agent treatment, or mutation, deletion or ligation using nucleic acid fragments produced by restriction enzyme cleavage, or the like. Such modified nucleic acids also fall within the scope of the invention, regardless of how different they may be from the base sequence shown in any of SEQ ID NOS:2 to 4, as long as they can hybridize to the base sequence shown in any of SEQ ID NOS:2 to 4 under stringent conditions and can produce proteins functionally equivalent in K-ras gene transcription-inhibiting activity to the protein encoded by the nucleic acid comprising the base sequence shown in any of SEQ ID NOS:2 to 4.

If the mutated sequence has a homology of 80% or more, preferably of 90% or more, with the base sequence shown in any of SEQ ID NOS:2 to 4, the degree of the mutation would be within the allowable range. The hybridization may be at such a level that hybridization to the nucleic acid defined in the sequence listing can be achieved by southern hybridization under usual conditions (for example, the conditions in which when probes are labeled with DIG DNA Labeling Kit (manufactured by Boehringer Mannheim), hybridization is performed in DIG Easy Hyb Solution (manufactured by Boehringer Mannheim) at 32° C., and the membrane is washed in a 0.5×SSC solution (containing 0.1% (w/v) SDS) at 50° C. (1×SSC is 0.15 M NaCl and 0.015 M sodium citrate)).

The nucleic acid of the invention may be used for the production of ESXR1-ΔC by recombination or the production of gene therapy vectors. Specifically, the nucleic acid of the invention is useful for the preparation of transformed cells, methods for producing ESXR1-ΔC with the transformed cells and gene therapy of cancers with ESXR1-ΔC. A vector, particularly a gene therapy virus vector, including a nucleic acid encoding ESXR1 and capable of providing ESXR1-ΔC in vivo may be used for gene therapy of cancers. Thus, such a vector also constitutes the invention.

Transformed cells having a gene encoding ESXR1-ΔC or ESXR1 may be prepared using techniques known to one skilled in the art. For example, the nucleic acid of the invention may be incorporated into appropriate host cells using a variety of commercially available vectors or vectors generally readily available to one skilled in the art. In such a process, the nucleic acid may be placed under the influence of an expression control gene such as a promoter and an enhancer so that the expression in the host cells can be controlled in the desired manner.

Nucleic Acid

The nucleic acid of the invention may be a single strand or may bind to a nucleic acid or RNA having a sequence complementary thereto to form a double or triple strand. The nucleic acid may also be labeled with an enzyme such as horse radish peroxidase (HRPO), a radioisotope, a fluorescent substance, a chemiluminescent substance, or the like.

The nucleic acid of the invention may be obtained from DNA libraries. Examples of such a technique include a method of screening human testis genome DNA or cDNA libraries by hybridization and a method including performing screening by immunoscreening method using an antibody or the like, amplifying a clone having the desired nucleic acid, and cutting the nucleic acid therefrom with a restriction enzyme or the like.

In the screening by hybridization, a nucleic acid having the base sequence shown in SEQ ID NO:2 or part thereof may be labeled with ³²P or the like to form a probe, and a known method may be performed with the probe on any cDNA library (for example, see Maniatis T. et al., Molecular Cloning, A Laboratory Manual, Cold Spring Harbor Laboratory, New York, 1982).

The immunoscreening method may use the antibody of the invention as described later. The nucleic acid of the invention may also be obtained by PCR (Polymerase Chain Reaction) using a genome DNA library or a cDNA library for templates. For example, sense and antisense primers may be prepared based on the base sequence shown in SEQ ID NO:1 or 2, and a known method may be performed with the primers on any DNA library so that the nucleic acid of the invention can be obtained (for example, see Michael A. I. et al., PCR Protocols, A Guide to Methods and Applications, Academic Press, 1990).

The DNA libraries for use in the various above-mentioned methods may be DNA libraries having the nucleic acid of the invention, and any human testis-derived library may be preferably used. Cells suitable for the preparation of cDNA libraries may also be selected from cells having the nucleic acid of the invention, and the cDNA libraries to be used may be prepared according to known methods (see J. Sambrook et al., Molecular Cloning, a laboratory Manual 2nd ed., Cold Spring Harbor Laboratory, New York, 1989).

Based on the sequences disclosed herein, the nucleic acid of the invention may also be prepared by chemical synthesis methods such as phosphoramidite methods.

Recombinant vectors having the nucleic acid of the invention may be in any form such as a circular form and a linear form. In addition to the nucleic acid of the invention, the recombinant vectors may have any other base sequence, if necessary. Examples of any other base sequence include enhancer sequences, promoter sequences, ribosome binding sequences, base sequences for use in amplifying the number of copies, signal peptide-encoding base sequences, base sequences encoding any other polypeptide, poly A addition sequences, splicing sequences, replication origins, and base sequences of selection marker genes.

In genetic recombination, any appropriate synthetic DNA adaptor may be used for the addition of a translation initiation codon or a translation termination codon to the nucleic acid of the invention or for new production or deletion of an appropriate restriction enzyme cleavage sequence in the base sequence. These falls within the routine work that one skilled in the art can usually perform, and based on the nucleic acid of the invention, processing can be readily performed in the desired manner by one skilled in the art.

Any appropriate vector may be selected and used to carry the nucleic acid of the invention, depending on the host to be used. While not only plasmids but also a variety of viruses such as bacteriophages, baculoviruses, retroviruses, and vaccinia viruses may be used, in particular, virus vectors developed for gene therapy are preferably used.

The gene of the invention may be expressed under the control of a promoter sequence specific to the gene. Any other appropriate expression promoter may be linked to or substituted for the gene-specific promoter sequence, upstream of the gene of the invention. In this case, any appropriate promoter may be selected and used, depending on the host and the purpose of the expression. Examples of the promoter include, but are not limited to, a T7 promoter, a lac promoter, a trp promoter, a λPL promoter and the like for E. coli hosts; a PHO5 promoter, a GAP promoter, an ADH promoter, and the like for yeast hosts; and an SV40-derived promoter, a retrovirus promoter and the like for animal cell hosts.

Known methods may be used to introduce the nucleic acid into vectors (see J. Sambrook et al., Molecular Cloning, A laboratory Manual 2nd ed., Cold Spring Harbor Laboratory, New York, 1989). Specifically, the nucleic acid and the vector may be each digested with an appropriate restriction enzyme, and the resulting fragments may be each ligated with a DNA ligase.

Protein

While the protein of the invention may be prepared from human testes, it is preferably prepared by a chemical synthesis method using a peptide synthesizer (for example, Peptide Synthesizer 430A manufactured by PerkinElmer Japan Co., Ltd.) or by a recombination method using appropriate host cells selected from prokaryotes and eukaryotes, in view of the purity and yield of the products.

Any host cell may be transformed with the recombinant vector without particular limitation, and in the invention, many types of cells may be used, such as lower cells available for genetic engineering, such as E. coli, B. subtilis and S. cerevisiae, insect cells, and animal cells such as COS7 cells, CHO cells and Hela cells.

Examples of methods for introducing the recombinant vector into host cells include electroporation techniques, protoplast techniques, alkali metal techniques, calcium phosphate precipitation techniques, DEAE dextran techniques, microinjection methods, and methods with virus particles. Any of these methods may be used.

A process for producing the protein by genetic engineering may include culturing the transformant, recovering the culture mixture, and purifying the protein. The transformant may be cultured by any general method.

Any appropriate method selected from the methods generally used for protein purification may be used to purify the protein of the invention from the culture mixture. Specifically, appropriate methods may be selected from general methods such as salting out, ultrafiltration, isoelectric precipitation, gel filtration, electrophoresis, ion-exchange chromatography, hydrophobic chromatography, various types of affinity chromatography such as antibody chromatography, chromatofocusing, adsorption chromatography, and reverse phase chromatography, and performed in an appropriate order for purification, optionally using an HPLC system or the like.

The protein of the invention may also be expressed in the form of a fusion protein with any other protein or tag (such as glutathione-S-transferase, protein A, a hexa-histidine tag, and an FLAG tag). The expressed fusion form may be digested with an appropriate protease (such as thrombin) so that the preparation of the protein can be more advantageously achieved in some cases. The protein of the invention may be purified by any appropriate combination of methods familiar with one skilled in the art. Particularly when the protein is expressed in the form of a fusion protein, purification methods specific to the form are preferably used.

A cell-free synthesis method using the recombinant DNA molecule (see J. Sambrook et al., Molecular Cloning 2nd ed. (1989)) is also one of the methods for producing the protein by genetic engineering.

As described above, the protein of the invention may be prepared in the form of a single protein by itself or in the form of a fusion protein with a different type of protein. However, the protein of the invention is not limited to these forms and may also be converted into various forms. For example, the protein may be processed by various techniques known to one skilled in the art, such as various types of chemical modification to proteins, coupling to polymers such as polyethylene glycol, coupling to insoluble carriers, and encapsulation into liposomes.

The transcriptional inhibitor for K-ras of the invention may be administered to a patient or a cancer tissue of a patient directly, singly or in combination with any appropriate vehicle and/or additive. For example, the transcriptional inhibitor of the invention may be encapsulated into an appropriate liposome, and the liposome may be delivered directly to cancer tissues.

The invention is more specifically described below by showing non-limiting examples. In the examples below, restriction enzyme treatment and the use of other commercially available enzymes or kits are all performed under the recommended conditions of reaction, unless otherwise stated.

### EXAMPLE 1

1) Preparation of Myc-Tag-Attached ESXR1 and ESXR1-ΔC Expression Vector

Oligonucleotides 1 and 2 were synthesized. Oligonucleotide 1 encodes the Myc-epitope and the amino acid residues 1 to 11 of the N-terminal of ESXR1 and has the sequence below. Oligonucleotide 2 has the sequence below that is complementary to a nucleic acid encoding the amino acid residues 232 to 241 of the C-terminal of ESXR1.

Oligonucleotide 1:
5′-CGGGATCCGCCGCCATGGAGCAAAAGCTCATTTCTGAAGAGGACTTG
AACGACTCTCTTCGCGGGTACACCCACAGTGAT-3′
Oligonucleotide 2:
5′-TAGTTGTGGCACCAGATGAACACACAAAGC-3′

The ESXR1 gene cloned by the method of Ozawa et al. (Ozawa et al., Oncogene, Vol. 23, 6590-6602, 2004) was used as a template, and PCR (50 μl in total) was performed under the conditions below using Takara ExTaq Kit (Takara), so that a DNA fragment encoding ESXR1 with the Myc-tag attached to the N-terminal was prepared.

| | | |
|---|---|---|
| Water | 38 | μl |
| Oligonucleotide 1 | 1 | μl (10 μM) |
| Oligonucleotide 2 | 1 | μl (10 μM) |
| dNTP mix | 4 | μl (2.5 mM) |
| 10 × PCR buffer | 5 | μl |
| ExTaq | 0.25 | μl |
| Template DNA | 1 | μl (1 μg/μl) |

Reaction cycle: 30 cycles of 30 seconds at 94° C., 30 seconds at 55° C. and 1 minute at 72° C.

The DNA fragment amplified by the PCR and a mammal expression vector pcDNA3 (Invitrogen) were digested with restriction enzymes BamHI (New England Biolabs (NEB)) and XbaI (NEB). A 1509-base-pair (bp) fragment resulting from the digestion and the open circular pcDNA3 were ligated with a DNA ligase (Takara), and pcDNA3/Myc-ESXR1 was prepared using E. coli DH5α strain as a host. A 731-bp fragment obtained by digesting the amplified DNA fragment with a restriction enzyme BamHI (NEB) and the open circular pcDNA3 obtained by digestion with the same restriction enzyme were ligated with a DNA ligase (Takara), and pcDNA3/Myc-ESXR1-ΔC was prepared using E. coli DH5α strain as a host.

2) Preparation of Cell Lines U2/tetESXR1 and U2/tetΔC

An opened expression vector pOPTET-BSD obtained by digestion with a restriction enzyme EcoRI (NEB) and by blunting with DNA Blunting Kit (Takara) (Hatakeyama et al., Anal. Biochem., 261, 211-218, 1998; Proc. Natl. Acad. Sci. USA, 95, 8574-8579, 1998) and the two types of vectors prepared in the section 1) were each digested with restriction enzymes HindIII (NEB) and XbaI (NEB) or digested with restriction enzymes KpnI (NEB) and XbaI (NEB) The recovered fragments resulting from the former digestion and encoding Myc-ESXR1 (SEQ ID NO:3) and the recovered fragments resulting from the latter digestion and encoding Myc-ESXR1-ΔC (SEQ ID NO:4) were each blunted with DNA Blunting Kit (Takara) and then ligated with a DNA ligase, and pOPTET-BSD/Myc-ESXR1 and pOPTET-BSD/Myc-ESXR1-ΔC were prepared using E. coli strain DH5α as a host.

U2-OS human osteosarcoma cell-derived Tet-on cells (CLONTECH) were transformed with each of these plasmid vectors by calcium phosphate transfection (Hinds, P. W. et al., Cell, 70, 993-1006, 1992). The transformed cells were then selected using a 10 μg/ml blasticidin-containing DMEM medium so that cell lines inducing and expressing Myc-ESXR1 or Myc-ESXR1-ΔC in a tetracycline-dependent manner were established as U2/tetESXR1 or U2/tetΔC.

3) Determination of ESXR1 Recognition Sequence

An M13 vector-derived forward primer sequence (M13-20), a restriction enzyme BamHI recognition sequence, a P3 consensus sequence recognizable by paired-like homeodomain, a restriction enzyme XbaI recognition sequence, and an M13-derived reverse primer sequence (M13 reverse) were ligated in the order from the 5′ end to the 3′ end so that 59-mer Oligonucleotide 3 as shown below was synthesized. The oligonucleotide was then converted into a double stranded DNA with ExTaq Polymerase (Takara).

Oligonucleotide 3:
5′-GTAAAACGACGGCCAGT-GGATCC-TAATNNNATTA-TCTAGA-CAT
GGTCATAGCTGTTTCC-3′

A synthetic DNA having a base sequence encoding the amino acid sequence of residues 139 to 198 of ESXR1 was inserted into a specific site of a vector PGEX 4T-2 (Amershambiosciences) for forming a fusion protein with glutathione-S-transferase (GST). Thereafter, under the recommended conditions, a GST-ESXR1 homeodomain fusion protein (GST-ESXR1HD) was induced and then purified. Thereafter, 0.4 μg of the resulting GST-ESXR1HD and 5 μg of the double stranded DNA were mixed in the presence of glutathione sepharose 4B beads (Pharmacia Biotech) in a 50 mM Tris-hydrochloric acid buffer (sonication buffer) (pH 8.0, containing 50 mM of NaCl, 1 mM of EDTA, 5 mM of DTT, 1 mM of PMSF, 10 μg/μl of leupeptin, 10 μg/μl of aprotinin, and 10 μg/μl of trypsin inhibitor) and a buffer containing 1% of Triton X-100 and 0.1% of bovine serum albumin (BSA) (Sigma) to form a protein-DNA complex. Thereafter, the beads were washed with the sonication buffer and a buffer containing 1% Triton X-100, 0.1% BSA, and 0.1% NP-40.

The beads were then suspended in 49.5 μl of the solution shown below and treated at 100° C. for 5 minutes. The beads were removed from the solution, and 0.5 μl of ExTaq was added to the resulting solution. PCR was then performed as described below.

| | | |
|---|---|---|
| Water | 29.5 | μl |
| Oligonucleotide 4: | 5 | μl |
| 5′-GTAAAACGACGGCCAGT-3′ | | |
| (10 pmol) | | |
| Oligonucleotide 5: | 5 | μl |
| 5′-GGAAACAGCTATGACCATG-3′ | | |
| (10 pmol) | | |
| dNTP mix | 5 | μl |
| 10XPCR buffer | 5 | μl |

Reaction cycle: incubation at 94° C. for 1 minutes followed by 10 to 20 cycles of 30 seconds at 94° C., 30 seconds at 70° C. and 60 seconds at 72° C.

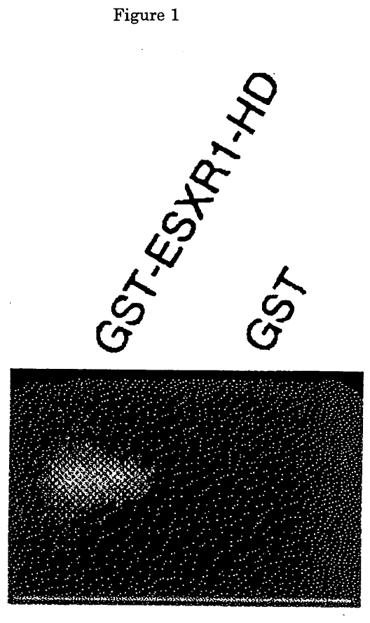

The DNA amplified by the PCR was digested with restriction enzymes BamHI and XbaI (each NEB). A 17 bp fragment resulting from the digestion was isolated and then mixed with the GST-ESXR1HD fusion protein under the same conditions as described above to form a protein-DNA complex, which was recovered using glutathione sepharose 4B beads. In total, 8 cycles of this process were performed, and then the DNA fragment amplified by PCR was cloned using pBluescript Vector (Stratagene), and the DNA base sequence was determined (FIG. 1). As a result, it has been found that the ESXR1 homeodomain specifically binds to the P3 consensus region comprising TAATNNNATTA (wherein N represents any nucleotide).

4) Electrophoretic Mobility Shift Assay

Based on the result of the section 3), an oligonucleotide having the base sequence shown below and Oligonucleotide 6 complementary thereto were each synthesized and formed into a double stranded probe.

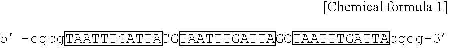

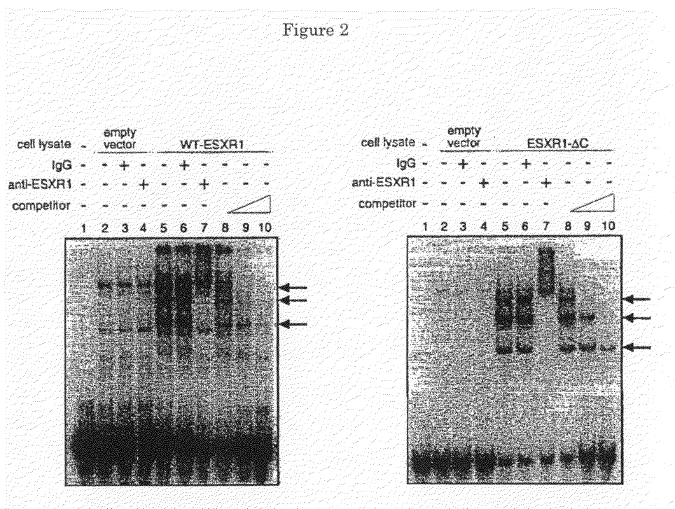

The probe was radiolabeled using [(α-³²P]dCTP (3,000 μCi/mmol, Amersham Biosciences) and Klenow DNA polymerase (Takara). COS-7 cells (1.5×10⁶) were transfected with 20 μg of each of the pcDNA3/Myc-ESXR1 and pcDNA3/Myc-ESXR1-ΔC prepared in the section 1) by calcium phosphate transfection. After cultured in a DMEM medium for 40 hours, the transformed cells were recovered, and 10 μg of the cell extract was mixed with 8 μl of a binding buffer (10 mM of HEPES, pH 7.5, 60 mM of NaCl, 4 mM of MgCl₂, 0.1 mM of EDTA, 0.1% of NP-40, and 10% of glycerol), 1 μg of poly(dI-dC), 10 μg of BSA, and 0.02 pmol of the radiolabeled double stranded probe to form 20 μl (total) of a reaction liquid, with which DNA binding reaction was performed on ice. After the reaction, the DNA-protein complex was subjected to electrophoresis at 200 V using a 0.5×Tris-boric acid-EDTA (TBE) electrophoresis buffer and 4% polyacrylamide gel. After the electrophoresis, the gel was analyzed by autoradiography (FIG. 2).

5) Luciferase Assay

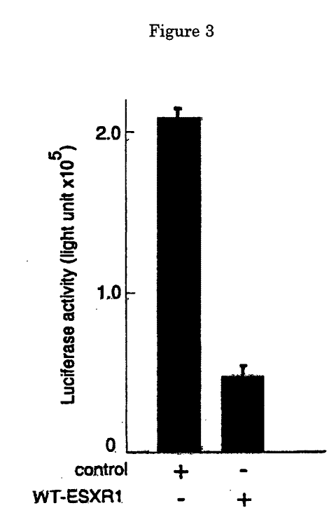

The double stranded probe prepared in the section 4) was inserted into the cloning site of a luciferase reporter plasmid pGL3-Promoter Vector (Promega) to form a recombinant reporter plasmid. 2 μg of the reporter plasmid and 20 μg of pcDNA3/Myc-ESXR1 were introduced into U2-OS osteosarcoma cells by calcium phosphate transfection. The cells were cultured in a DMEM medium for 12 hours and then recovered, and the luciferase activity of the cell extract was measured by the recommended method. As a result, it was found that in the cells expressing Myc-ESXR1, the expression of the luciferase was inhibited specifically to the base sequence obtained in the section 4) (FIG. 3).

6) Determination of Gene Whose Transcription is Inhibited by the Expression of ESXR1

DOX was added at a concentration of 2 μg/ml to 3.0×10⁶ U2tetESXR1 cells cultured in a DMEM medium for 24 hours, so that the expression of Myc-ESXR1 was induced. After 12 hours, the cells were recovered. The cells untreated with DOX were used as a control.

Total RNA was extracted from the cells with Trizol Reagent (GIBCO), and the purity of the RNA was checked with a formaldehyde-modified gel. According to the protocol recommended by Affymetrix, 5 μg of the total RNA was reverse-transcribed into cDNA using SuperScript II (Invitrogen). In the preparation of the cDNA, Oligonucleotide 7 having the sequence below (Amersham BioSciences) was used as a primer.

5′-GGCCAGTGAATTGTAATACGACTCACTATAGGGAGGCGG-
(T) 24-3′

The cDNA was turned into a double strand, which was purified with Phase Lock Gel (Eppendorf). The in vitro transcription reaction was performed using Enzo BioArray High Yield RNA Transcript Labeling Kit (Enzo Diagnostics) and 1 μg of cDNA. The resulting cRNA was purified using RNeasy Clean-Up columns (Qiagen) and then fragmented by heating in a 40 mM Tris-acetate buffer, pH 8.1, containing 100 mM of KOAc and 30 mM of MgOAc.

10 μg of the fragmented cRNA was subjected to hybridization (45° C., 16 hours) in a hybridization buffer containing 100 mM of MES, 1M of NaCl, 20 mM of EDTA, and 0.01% of Tween-20. According to the EukGE-WS2v4 protocol, cleaning and staining were performed using Fluidics Station 400 (Affymetrix). Chip data were scanned using Affymetrix GeneChip Scanner 3000 (Affymetrix). The analysis of chips was performed using Affymetrix GeneChip (registered trademark) Operating Software Ver1.1 (Affymetrix).

Based on the resulting DNA chip analysis data, existing genes whose transcription was inhibited by the expression of ESXR1 and which had the TAATNNNATTA sequence (wherein N is any base) in the genome sequence were selected. As a result, a K-ras gene having a P3 consensus sequence of TAATGTTATTA in the first intron base sequence was identified as a target gene candidate for the inhibition of transcription by ESXR1.

7) Inhibition of the Expression of K-Ras by ESXR1

After 5.0×10⁵ U2/tetESXR1 cells were cultured in a DMEM medium for 24 hours, doxycycline (DOX) was added at 2 μg/ml medium to induce the expression of ESXR1.

Twelve hours after the addition of DOX, the cells were recovered and lysed using an E1A buffer, and then the protein concentration was measured. Using 150 μg of the protein, 15% polyacrylamide gel electrophoresis was performed. The proteins separated on the gel were transferred to a polyvinylidene difluoride (PVDF) filter (Millipore), and western blotting was performed using a K-Ras protein-recognizing monoclonal antibody. The cells without the addition of DOX were used as control cells, and an anti-actin antibody was used for the internal control.

The proteins were detected using ECL Detection System (Perkin Elmer), and the protein bands were quantified using a luminescent image analyzer (LAS-100 manufactured by Fujifilm Corporation).

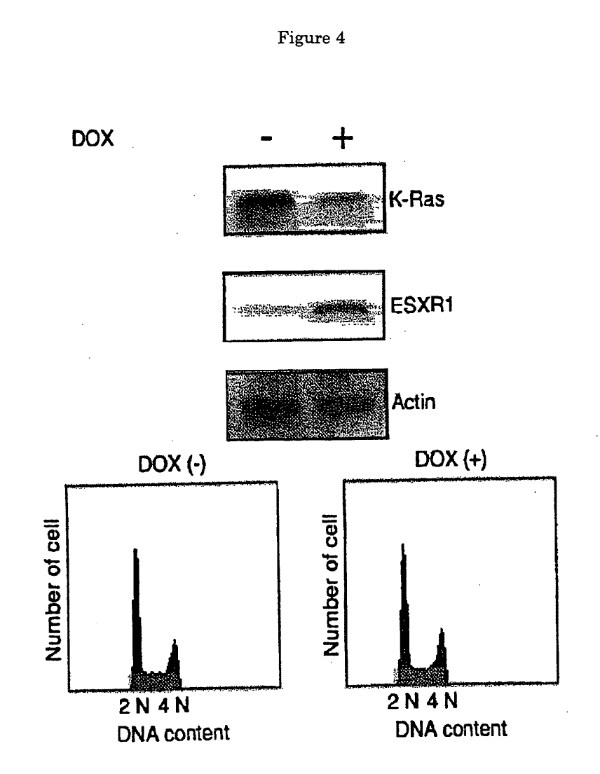

As a result, a significant reduction in the K-Ras protein level in the U2/tetESXR1 cells was observed as ESXR1 was induced and expressed (FIG. 4).

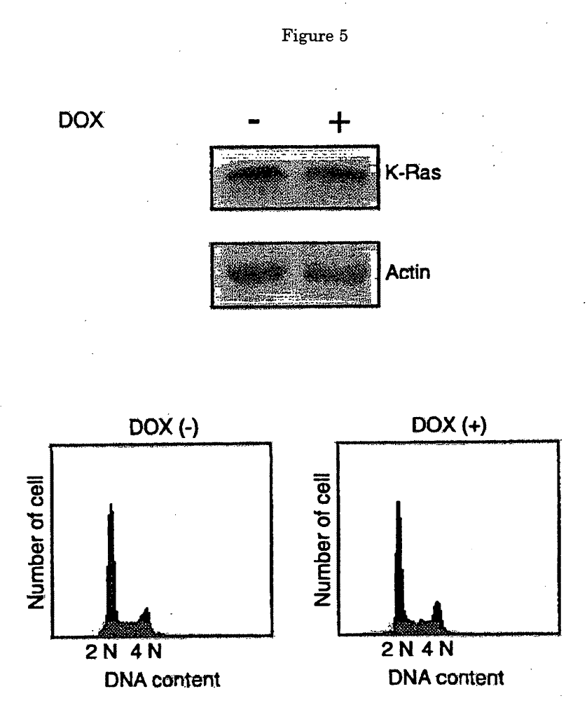

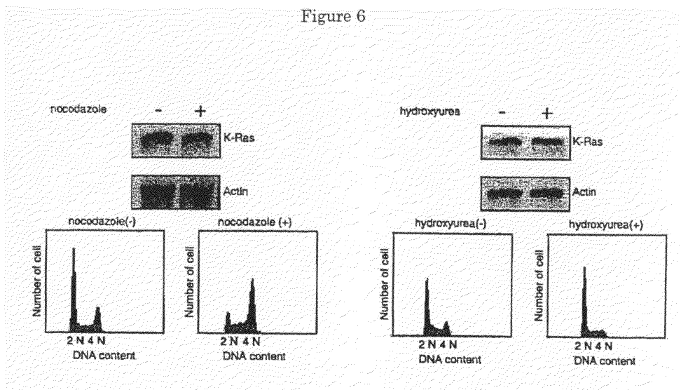

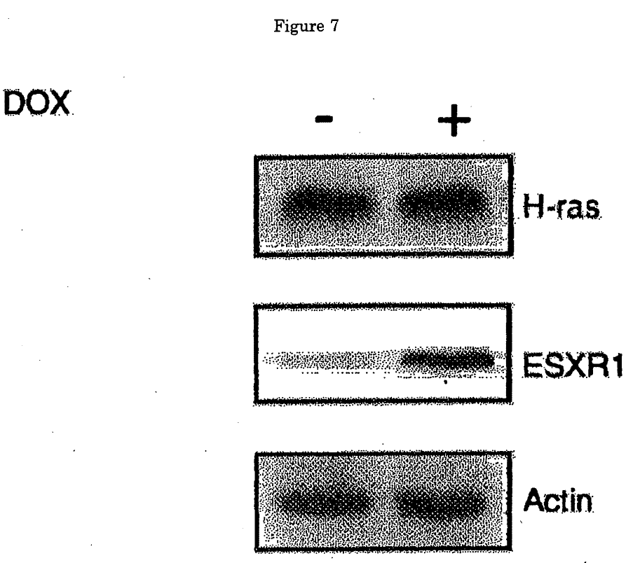

On the other hand, U2-OS cells (parent strain) were used for the comparison of the K-Ras protein expression, in order to determine the influence of DOX on the K-Ras expression. As a result, there was not observed any DOX-induced change in the K-Ras protein (FIG. 5). In addition, hours after the DOX induction, there was no influence on the cell cycle in the U2/tetESXR1 cells (FIG. 4). However, it is known that ESXR1 can induce M-phase cell cycle arrest. Thus, the K-Ras expression was compared when the U2/tetESXR1 cells were arrested at the S or G2/M phase by treatment with 5 mM of hydroxyurea and 50 μM of nocodazole. As a result, no cell cycle-dependent change in the K-Ras protein expression was observed (FIG. 6). Thus, the expression of H-Ras which is a molecule of the same Ras family was examined in order to determine whether or not the ESXR1-induced reduction in the K-Ras protein expression was specific to K-Ras. As a result, the ESXR1 expression had no influence on the H-Ras protein level (FIG. 7). The foregoing has demonstrated that ESXR1 specifically inhibits K-Ras expression in cells.

8) Analysis of K-ras mRNA Expression by Real Time PCR

Total RNA was extracted from 3.0×10⁶ U2/tetESXR1 cells or U2/tetΔC cells using Trizol (GIBCO). SuperScript II reverse transcriptase (Invitrogen) was used to synthesize cDNA from 10 μg of the total RNA, and under the conditions below, real time PCR was performed using SYBRgreen PCR Kit (Strategene) and ABIPRISM 7700 Sequence Detector (Perkin Elmer).

For the PCR, the following reaction liquid (a total amount of 13.6 μl) was first prepared.

Oligonucleotide 8: 5′-CCAGGTGCGGGAGAGAG-3′
Oligonucleotide 9: 5′-CCCTCATTGCACTGTACTCC-3′

| | | |
|---|---|---|
| Total RNA solution (1 mg/ml) | 10 | μl |
| Oligo (dT)₁₂₋₁₈ solution (500 μg/ml) | 1 | μl |
| DEPC-treated milli-Q water | 2.6 | μl |

The above reaction liquid was heated at 70° C. for 10 minutes and then allowed to stand on ice for 1 minute. Thereafter, 6.4 μl of a premix having the composition below was added to the reaction liquid.

| | |
|---|---|
| 5x First strand buffer (250 mM Tris-hydrochloric | 4 μl |
| acid, pH 8.3, 375 mM of KCl, 15 mM of MgCl₂) | |
| 25 mM dNTP mix | 1 μl |
| 0.1 M DTT | 2 μl |

Thereafter, 1 μl (200 units) of SuperScript II reverse transcriptase was added thereto, thoroughly mixed and then allowed to stand at 42° C. for 50 minutes and at 70° C. for 15 minutes, respectively. Thereafter, 1 μl (2 units) of RNase H was added thereto and allowed to react at 37° C. for 20 minutes to decompose the template RNA so that a single-stranded cDNA was obtained. The cDNA was used to form the following liquid mixture (a total amount of 25 μl).

| 1 sample | | |
|---|---|---|
| SYBRgreenPCR Kit (Strategene) | 12.5 | μl |
| Forward Primer 8 (10 pmol/μl): | 1 | μl |
| 5′-CCAGGTGCGGGAGAGAG-3′ | | |
| Reverse Primer 9 (10 pmol/μl): | 1 | μl |
| 5′-CCCTCATTGCACTGTACTCC-3′ | | |
| cDNA | 1 | μl |
| MQ | 9.5 | μl |

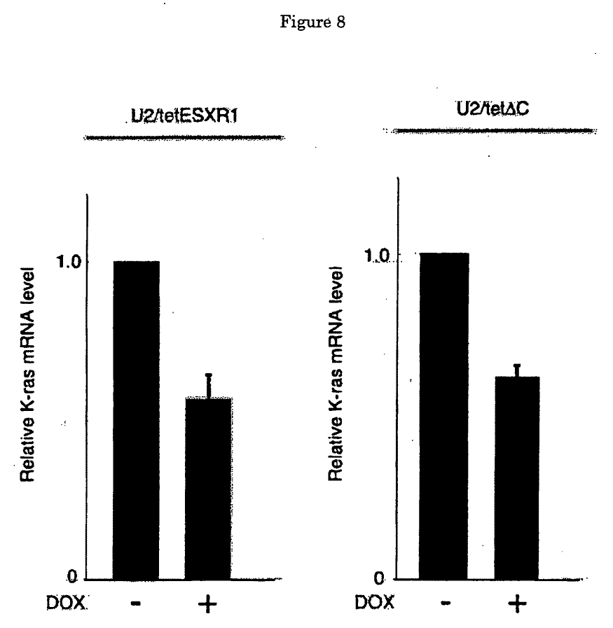

Using 20 μl of the reaction liquid, real time PCR was performed in ABIPRISM 7700 Sequence Detector (Perkin Elmer). The reaction included standing at 50° C. for 2 minutes and at 95° C. for 10 minutes, respectively, and then 40 cycles of 15 seconds at 95° C. and 1 minute at 60° C. As a result, it was observed that the expression of the K-ras mRNA was reduced by the induction of ESXR1 or ESXR1-ΔC in U2/tetESXR1 or U2/tetΔC cells (FIG. 8). In order to further verify the result, a reporter vector was prepared in which Oligonucleotide 10 having the sequence below containing 5′-TAATGTTATTA-3′, which exists in the first intron of the K-ras gene, was inserted upstream of the SV40 promoter of a pGL3 promoter vector (Promega), and luciferase assay was performed.

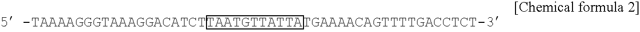

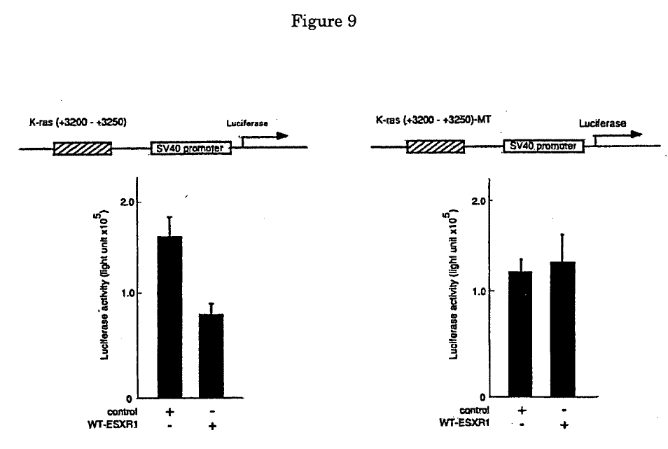

Also in this reporter assay, transcription inhibition for ESXR1-dependent luciferase was observed (FIG. 9). The result of the electrophoretic mobility shift assay has revealed that ESXR1 binds to the consensus sequence portion but does not bind to Oligonucleotide 11:

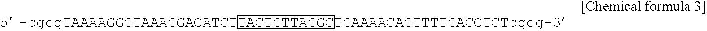

in which the consensus sequence is modified into the indicated portion.

9) Determination of Ability to Inhibit Cancer Cell Growth

Using Lipofectamine (trade mark) 2000 Reagent (Invitrogen), 19 μg of pcDNA3/Myc-ESXR1-ΔC and 1 μg of pBabe-puro vector were introduced into human colon cancer cells SW480 (4.0×10⁶) having an active K-ras gene mutation.

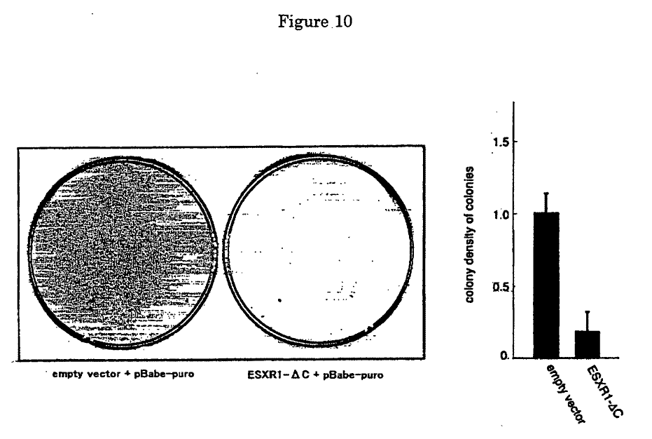

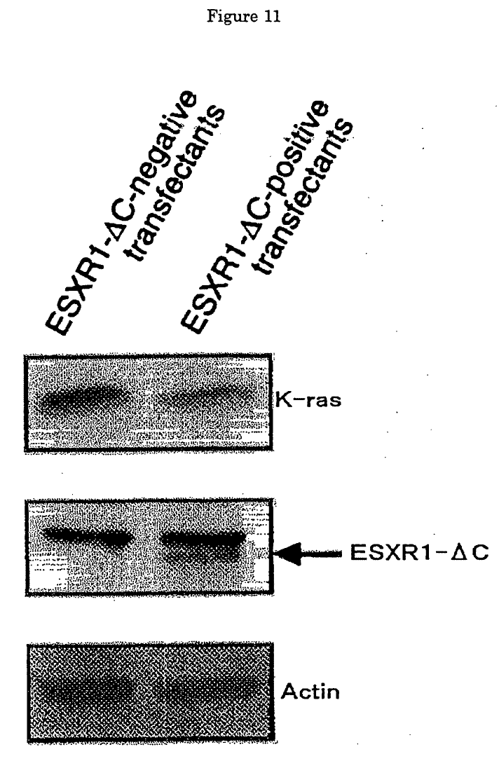

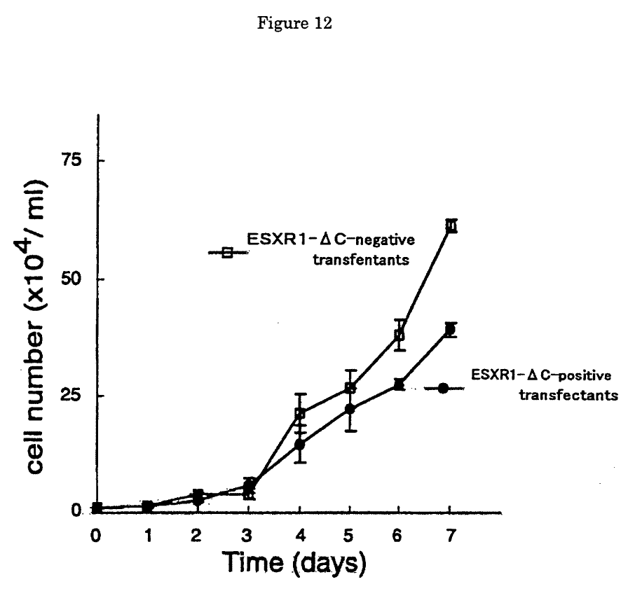

After cultured in a DMEM/F12 medium for 24 hours, the cells were diluted 10-fold and continued to be cultured, and 24 hours thereafter, 1.5 μg/ml of puromycin was added. The cells were further cultured for 3 weeks, and then drug-resistant cells were selected. SW480 cells transformed with 19 μg of pcDNA3 and 1 μg of pBabe-puro in the same manner were used as a control. The cells expressing ESXR1-ΔC showed a significant reduction of drug-resistant colonies as compared with the non-expressing cells (FIG. 10). In addition, pcDNA3/Myc-ESXR1-ΔC and pBabe-puro were introduced into colon cancer cells SW480 by calcium phosphate transfection, and the cells were selected using puromycin so that transfectant cells constitutively expressing ESXR1-ΔC were obtained. The cell group expressing ESXR1-ΔC showed a significant reduction in the K-Ras expression and a reduction in the cell proliferation as compared with the ESXR1-ΔC non-expressing cell group (FIGS. 11 and 12).

The result has revealed that ESXR1-ΔC inhibits the expression of the K-ras gene and reduces the K-Ras expression level so that it inhibits the growth of cancer cells in which the cancer trait is maintained by the mutated K-ras.

### INDUSTRIAL APPLICABILITY

The transcriptional inhibitor for K-ras gene of the invention can specifically inhibit the transcription and expression of the K-ras gene which is an oncogene in human cancer cells, so that it can induce inhibition of cancer cell growth and can also induce a return from cancer cells to normal cells. The transcriptional inhibitor for K-ras gene of the invention can also inhibit the expression of a normal K-ras gene. However, its function can be compensated by the expression of other genes of the Ras family. Thus, the inhibitor of the invention has no influence on normal cell growth or differentiation and thus can provide an anticancer agent with less side effects on normal cells.

1. A transcriptional inhibitor for human K-ras gene, comprising one or more proteins selected from the group consisting of a protein comprising an amino acid sequence shown in SEQ ID NO:1, a protein comprising an amino acid sequence derived from the amino acid sequence shown in SEQ ID NO:1 by substitution, deletion or addition of one or several amino acids and having an activity of inhibiting the transcription of human K-ras gene, or a protein fragment thereof having an activity of inhibiting the transcription of human K-ras gene.

2. The transcriptional inhibitor for human K-ras gene according to claim 1,
wherein the protein fragment is an N-terminal fragment with a molecular weight of 45 kD.

3. The transcriptional inhibitor for human K-ras gene according to claim 2,
wherein the protein fragment has an amino acid sequence of residues 1 to 229 of SEQ ID NO:1 or an amino acid sequence derived from the amino acid sequence of residues 1 to 229 by substitution, deletion or addition of one or several amino acids.

4. A protein comprising any one of the amino acid sequences shown in SEQ ID NOS: 2 to 4, or a protein derived from the protein comprising any one of the amino acid sequences shown in SEQ ID NOS: 2 to 4 by substitution, deletion or addition of one or several amino acids and having an activity of inhibiting the transcription of human K-ras gene.

5. A nucleic acid encoding the protein according to claim 4.

6. A nucleic acid comprising a base sequence shown in any one of SEQ ID NOS:2 to 4 or a nucleic acid comprising a base sequence capable of hybridizing to any one of the base sequences shown in SEQ ID NOS:2 to 4 under stringent conditions and encoding a protein having an activity of inhibiting the transcription of human K-ras gene.

7. A recombinant virus vector for use in gene therapy of cancer, comprising either a nucleic acid encoding the protein according to any one of claims 1 to 4 or the nucleic acid according to claim 5 or 6.

8. A recombinant vector, comprising the nucleic acid according to claim 5 or 6.

9. A method for treating cancer, comprising administering the transcriptional inhibitor for human K-ras gene according to any one of claims 1 to 3 or the protein according to claim 4 to cancer cells.

10. A method for gene therapy of cancer, comprising introducing the recombinant virus vector according to claim 7 into cancer cells of a patient.


SEQUENCE LISTING (23 sequences)

SEQ ID NO: 1 - protein, 406 aa, Homo sapiens
MESLRGYTHSDIGYRSLAVGEDIEEVNDEKLTVTSLMARGGEDEENTRSKPEYGTEAENNVGTEGSVPSDDQDREGGGGHEPEQQQEEPPLTKPEQQQEEPPLLELKQEQEEPPQTTVEGPQPAEGPQTAEGPQPPERKRRRRTAFTQFQLQELENFFDESQYPDVVARERLAARLNLTEDRVQVWFQNRRAKWKRNQRVLMLRNTATADLAHPLDMFLGGAYYAAPALDPALCVHLVPQLPRPPVLPVPPMPPRPPMVPMPPRPPIAPMPPMAPVPPGSRMAPVPPGPRMAPVPPWPPMAPVPPWPPMAPVPTGPPMAPVPPGPPMARVPPGPPMARVPPGPPMAPLPPGPPMAPLPPGPPMAPLPPGPPMAPLPPRSHVPHTGLAPVHITWAPVINSYYACPFF

SEQ ID NO: 2 - protein, 229 aa, Homo sapiens
MESLRGYTHSDIGYRSLAVGEDIEEVNDEKLTVTSLMARGGEDEENTRSKPEYGTEAENNVGTEGSVPSDDQDREGGGGHEPEQQQEEPPLTKPEQQQEEPPLLELKQEQEEPPQTTVEGPQPAEGPQTAEGPQPPERKRRRRTAFTQFQLQELENFFDESQYPDVVARERLAARLNLTEDRVQVWFQNRRAKWKRNQRVLMLRNTATADLAHPLDMFLGGAYYAAPAL

SEQ ID NO: 3 - protein, 417 aa, Artificial Sequence
  Description of Artificial Sequence Synthetic Myc-tag and ESXR1 fusion polypeptide
MEQKLISEEDLNESLRGYTHSDIGYRSLAVGEDIEEVNDEKLTVTSLMARGGEDEENTRSKPEYGTEAENNVGTEGSVPSDDQDREGGGGHEPEQQQEEPPLTKPEQQQEEPPLLELKQEQEEPPQTTVEGPQPAEGPQTAEGPQPPERKRRRRTAFTQFQLQELENFFDESQYPDVVARERLAARLNLTEDRVQVWFQNRRAKWKRNQRVLMLRNTATADLAHPLDMFLGGAYYAAPALDPALCVHLVPQLPRPPVLPVPPMPPRPPMVPMPPRPPIAPMPPMAPVPPGSRMAPVPPGPRMAPVPPWPPMAPVPPWPPMAPVPTGPPMAPVPPGPPMARVPPGPPMARVPPGPPMAPLPPGPPMAPLPPGPPMAPLPPGPPMAPLPPRSHVPHTGLAPVHITWAPVINSYYACPFF

SEQ ID NO: 4 - protein, 277 aa, Artificial Sequence
  Description of Artificial Sequence Synthetic Myc-tag and ESXR1dC fusion polypeptide
MEQKLISEEDLNESLRGYTHSDIGYRSLAVGEDIEEVNDEKLTVTSLMARGGEDEENTRSKPEYGTEAENNVGTEGSVPSDDQDREGGGGHEPEQQQEEPPLTKPEQQQEEPPLLELKQEQEEPPQTTVEGPQPAEGPQTAEGPQPPERKRRRRTAFTQFQLQELENFFDESQYPDVVARERLAARLNLTEDRVQVWFQNRRAKWKRNQRVLMLRNTATADLAHPLDMFLGGAYYAAPALDPLVTAASVLEFCRYPSHWRPLEHASRGPYSIVSPKC

SEQ ID NO: 5 - DNA, 80 bp, Artificial Sequence
  Description of Artificial Sequence Synthetic oligonucleotide
cgggatccgccgccatggagcaaaagctcatttctgaagaggacttgaacgagtctcttcgcgggtacacccacagtgat

SEQ ID NO: 6 - DNA, 30 bp, Artificial Sequence
  Description of Artificial Sequence Synthetic oligonucleotide
tagttgtggcaccagatgaacacacaaagc

SEQ ID NO: 7 - DNA, 59 bp, Artificial Sequence
  Description of Artificial Sequence Synthetic oligonucleotide
gtaaaacgacggccagtggatcctaatnnnattatctagacatggtcatagctgtttcc

SEQ ID NO: 8 - DNA, 17 bp, Artificial Sequence
  Description of Artificial Sequence Synthetic oligonucleotide
gtaaaacgacggccagt

SEQ ID NO: 9 - DNA, 19 bp, Artificial Sequence
  Description of Artificial Sequence Synthetic oligonucleotide
ggaaacagctatgaccatg

SEQ ID NO: 10 - DNA, 45 bp, Artificial Sequence
  Description of Artificial Sequence Synthetic oligonucleotide
cgcgtaatttgattacgtaatttgattagctaatttgattacgcg

SEQ ID NO: 11 - DNA, 63 bp, Artificial Sequence
  Description of Artificial Sequence Synthetic oligonucleotide
ggccagtgaattgtaatacgactcactatagggaggcggtttttttttttttttttttttttt

SEQ ID NO: 12 - DNA, 17 bp, Artificial Sequence
  Description of Artificial Sequence Synthetic oligonucleotide
ccaggtgcgggagagag

SEQ ID NO: 13 - DNA, 20 bp, Artificial Sequence
  Description of Artificial Sequence Synthetic oligonucleotide
ccctcattgcactgtactcc

SEQ ID NO: 14 - DNA, 59 bp, Artificial Sequence
  Description of Artificial Sequence Synthetic oligonucleotide
cgcgtaaaagggtaaaggacatcttaatgttattatgaaaacagttttgacctctcgcg

SEQ ID NO: 15 - DNA, 59 bp, Artificial Sequence
  Description of Artificial Sequence Synthetic oligonucleotide
cgcgtaaaagggtaaaggacatcttactgttaggctgaaaacagttttgacctctcgcg

SEQ ID NO: 16 - DNA, 1221 bp, Homo sapiens
  CDS (1)..(1221): ESXR1
atggagtctcttcgcgggtacacccacagtgatattggctaccgcagcctggcagtcggcgaggacatcgaggaagtgaatgatgagaaacttaccgtgacctcgctgatggcaaggggaggagaggacgaggagaatacacggtccaaacctgagtacggaacagaagcggaaaacaacgttggcacggaagggtccgtcccctcggacgaccaagaccgtgagggtggcggcggccacgagccggagcaacagcaggaggagccgcccctgaccaagccggagcaacagcaggaggagccgcccctgctcgagctgaagcaagagcaggaggagccgccccagacgaccgtggaggggccacagccggcggaggggccacaaaccgctgagggaccacagcccccagagaggaaacgccgccgccgcaccgcgttcacgcagtttcagctgcaggagctagagaactttttcgatgaatctcaatatcccgacgttgtggcgcgagagagacttgcagcacgcctgaatttgactgaagacagagtgcaggtttggtttcagaacagaagagccaagtggaaacgaaatcagagggtgctaatgttgagaaacactgctactgctgacctggcccaccctttggacatgttcttgggtggggcctattatgctgctcctgctctggatcctgctttgtgtgttcatctggtgccacaactacctagaccacctgtgctgcctgtgccacctatgccacccaggccacccatggtccctatgccacccaggccacccatagcacctatgccacccatggcgcctgtgccacccggctcacgcatggcgcctgtgccacccgggccacgcatggcgcctgtgccaccctggccgcccatggcgcctgtgccaccctggccgcccatggcgcctgtgccaaccgggccgcccatggcgcctgtgccacccgggccgcccatggcgcgtgtgccacccgggccgcccatggcgcgtgtgccacccgggccgcccatggcgcctctgccacccgggccgcccatggcgcctctgccacccgggccgcccatggcgcctctgccacccgggccgcccatggcgcctctgccacccaggtcacatgtgcctcacactggcctggctcctgtacacatcacatgggcccctgtcatcaacagttattatgcatgtccctttttctaa

SEQ ID NO: 17 - DNA, 687 bp, Homo sapiens
  CDS (1)..(687): ESXR1dC
atggagtctcttcgcgggtacacccacagtgatattggctaccgcagcctggcagtcggcgaggacatcgaggaagtgaatgatgagaaacttaccgtgacctcgctgatggcaaggggaggagaggacgaggagaatacacggtccaaacctgagtacggaacagaagcggaaaacaacgttggcacggaagggtccgtcccctcggacgaccaagaccgtgagggtggcggcggccacgagccggagcaacagcaggaggagccgcccctgaccaagccggagcaacagcaggaggagccgcccctgctcgagctgaagcaagagcaggaggagccgccccagacgaccgtggaggggccacagccggcggaggggccacaaaccgctgagggaccacagcccccagagaggaaacgccgccgccgcaccgcgttcacgcagtttcagctgcaggagctagagaactttttcgatgaatctcaatatcccgacgttgtggcgcgagagagacttgcagcacgcctgaatttgactgaagacagagtgcaggtttggtttcagaacagaagagccaagtggaaacgaaatcagagggtgctaatgttgagaaacactgctactgctgacctggcccaccctttggacatgttcttgggtggggcctattatgctgctcctgctctg

SEQ ID NO: 18 - DNA, 1254 bp, Artificial Sequence
  Description of Artificial Sequence Synthetic Myc-tag and ESXR1 fusion polynucleotide
atggagcaaaagctcatttctgaagaggacttgaacgagtctcttcgcgggtacacccacagtgatattggctaccgcagcctggcagtcggcgaggacatcgaggaagtgaatgatgagaaacttaccgtgacctcgctgatggcaaggggaggagaggacgaggagaatacacggtccaaacctgagtacggaacagaagcggaaaacaacgttggcacggaagggtccgtcccctcggacgaccaagaccgtgagggtggcggcggccacgagccggagcaacagcaggaggagccgcccctgaccaagccggagcaacagcaggaggagccgcccctgctcgagctgaagcaagagcaggaggagccgccccagacgaccgtggaggggccacagccggcggaggggccacaaaccgctgagggaccacagcccccagagaggaaacgccgccgccgcaccgcgttcacgcagtttcagctgcaggagctagagaactttttcgatgaatctcaatatcccgacgttgtggcgcgagagagacttgcagcacgcctgaatttgactgaagacagagtgcaggtttggtttcagaacagaagagccaagtggaaacgaaatcagagggtgctaatgttgagaaacactgctactgctgacctggcccaccctttggacatgttcttgggtggggcctattatgctgctcctgctctggatcctgctttgtgtgttcatctggtgccacaactacctagaccacctgtgctgcctgtgccacctatgccacccaggccacccatggtccctatgccacccaggccacccatagcacctatgccacccatggcgcctgtgccacccggctcacgcatggcgcctgtgccacccgggccacgcatggcgcctgtgccaccctggccgcccatggcgcctgtgccaccctggccgcccatggcgcctgtgccaaccgggccgcccatggcgcctgtgccacccgggccgcccatggcgcgtgtgccacccgggccgcccatggcgcgtgtgccacccgggccgcccatggcgcctctgccacccgggccgcccatggcgcctctgccacccgggccgcccatggcgcctctgccacccgggccgcccatggcgcctctgccacccaggtcacatgtgcctcacactggcctggctcctgtacacatcacatgggcccctgtcatcaacagttattatgcatgtccctttttctaa

SEQ ID NO: 19 - DNA, 834 bp, Artificial Sequence
  Description of Artificial Sequence Synthetic Myc-tag and ESXR1dC fusion polynucleotide
atggagcaaaagctcatttctgaagaggacttgaacgagtctcttcgcgggtacacccacagtgatattggctaccgcagcctggcagtcggcgaggacatcgaggaagtgaatgatgagaaacttaccgtgacctcgctgatggcaaggggaggagaggacgaggagaatacacggtccaaacctgagtacggaacagaagcggaaaacaacgttggcacggaagggtccgtcccctcggacgaccaagaccgtgagggtggcggcggccacgagccggagcaacagcaggaggagccgcccctgaccaagccggagcaacagcaggaggagccgcccctgctcgagctgaagcaagagcaggaggagccgccccagacgaccgtggaggggccacagccggcggaggggccacaaaccgctgagggaccacagcccccagagaggaaacgccgccgccgcaccgcgttcacgcagtttcagctgcaggagctagagaactttttcgatgaatctcaatatcccgacgttgtggcgcgagagagacttgcagcacgcctgaatttgactgaagacagagtgcaggtttggtttcagaacagaagagccaagtggaaacgaaatcagagggtgctaatgttgagaaacactgctactgctgacctggcccaccctttggacatgttcttgggtggggcctattatgctgctcctgctctggatccactagtaacggccgccagtgtgctggaattctgcagatatccatcacactggcggccgctcgagcatgcatctagagggccctattctatagtgtcacctaaatgctag

SEQ ID NO: 20 - DNA, 11 bp, Artificial Sequence
  Description of Artificial Sequence Synthetic oligonucleotide
taatgttatta

SEQ ID NO: 21 - protein, 5 aa, Artificial Sequence
  Description of Artificial Sequence Synthetic Hexa-His tag
HHHHH

SEQ ID NO: 22 - DNA, 11 bp, Artificial Sequence
  Description of Artificial Sequence Synthetic oligonucleotide
taatnnnatta

SEQ ID NO: 23 - DNA, 51 bp, Artificial Sequence
  Description of Artificial Sequence Synthetic oligonucleotide
taaaagggtaaaggacatcttaatgttattatgaaaacagttttgacctct